(19) United States
(12) Patent Application Publication
(10) Pub. No.: US 2026/0283471 A1
(43) Pub. Date: Sep. 24, 2026

(54) HYPERSPECTRAL MEDICAL IMAGING PLATFORM AND METHODS

(71) Applicant: SpectraServe LLC, St. Louis, MO (US)

(72) Inventors: Michael B. Wong, Philadelphia, PA (US); Michael A. de Jong, Boston, MA (US)

(21) Appl. No.: 19/475,138

(22) PCT Filed: Apr. 11, 2024
(86) PCT No.: PCT/US2024/024029

§ 371 Date: Oct. 14, 2025

Related U.S. Application Data
(60) Provisional application No. 63/496,354, filed on Apr. 14, 2023.

(51) Int. Cl. A61B 5/00; G06V 10/143; G06V 10/147; G06V 10/25; G06V 10/762; G06V 10/764; H04N 23/11; H04N 23/12; H04N 23/56

(52) U.S. Cl. CPC A61B 5/0075; A61B 5/0037; A61B 5/742; G06V 10/143; G06V 10/147; G06V 10/25; G06V 10/762; G06V 10/764; H04N 23/11; H04N 23/12; H04N 23/56; A61B 2576/00; G06V 2201/03

(57) ABSTRACT

Systems and methods for generating hyperspectral medical images capable of producing and processing hyperspectral images of in vivo or ex vivo tissue samples. In processing hyperspectral images, the systems and methods are capable of producing pseudocolor or color-coded images, indicating various tissue types present in the image. The systems and methods may be especially useful for surgical margin assessment, including intraoperative surgical margin assessment, for example, for determining surgical margins during tumor resection.

### CROSS-REFERENCES TO RELATED APPLICATIONS

This application claims benefit of U.S. Provisional Application No. 63/496,354 filed Apr. 14, 2023, the specification of which is incorporated herein in its entirety by reference.

### FIELD OF THE INVENTION

The present invention relates to the fields of medical imaging and hyperspectral imaging. Particularly, the present invention relates to the intersection of these two fields.

### BACKGROUND OF THE INVENTION

Hyperspectral imaging relates broadly to the collection and processing of information from across the electromagnetic (EM) spectrum. In general, the goal of hyperspectral imaging is to obtain an electromagnetic spectrum for each pixel in an image. Such an image may be used to find objects, identify materials, or detect processes.

The human eye perceives electromagnetic radiation in three bands. These three bands form what is known as the visible light spectrum, or simply the visible spectrum. The visible spectrum ranges from about 380 to about 700 nanometers (nm). Longer wavelengths within the visible spectrum appear red, medium wavelengths appear green, and shorter wavelengths within the visible spectrum appear blue. Hyperspectral imaging, on the other hand, divides the electromagnetic spectrum into many more bands, some of which can extend beyond the visible spectrum. Thus, hyperspectral imaging may detect bands with wavelengths shorter than those in the visible spectrum (i.e., the ultraviolet region and wavelengths shorter than ultraviolet) and/or may detect bands with wavelengths longer than those in the visible spectrum (i.e., the infrared region and wavelengths longer than infrared).

Additionally, the wavelength bands typically used in hyperspectral imaging are more numerous, and/or more narrow than the wavelength bands perceived by the human eye and many other imaging systems. This allows for a more precise representation of the electromagnetic reflectance, transmission, or emission spectrum of a given object.

Imaged objects often have unique spectral “fingerprints” known as spectral signatures. A spectral signature is the variation in wavelengths reflected from, emitted by, or transmitted through a material. As it relates to reflected electromagnetic radiation, the spectral signature of an object is a function of the wavelength of incident electromagnetic radiation and the object's material interaction with that portion of the electromagnetic spectrum. Spectral signatures, because they are unique to a given material, can be used to identify materials. For example, the spectral signature of oil may be used to map the extent of an oil spill.

Hyperspectral imaging is accomplished using hyperspectral sensors. Hyperspectral sensors collect information as a set of “images,” each of which represents a spectral band (i.e., a relatively narrow wavelength range of the electromagnetic spectrum). In some applications, these “images” may be combined to form a 3D (x, y, A) hyperspectral data cube, wherein x and y are the two spatial dimensions of the imaged scene, and A is the spectral dimension (i.e., a range of wavelengths).

The precision of hyperspectral imaging may be measured in one of two primary ways. First, spectral resolution describes the width of each band of the spectrum that is captured. In other words, spectral resolution may be thought of as the minimum wavelength (or frequency) difference between two lines in a spectrum that can be distinguished from one another. Second, spatial resolution deals with the size of each pixel in the hyperspectral image. For example, pixels that are very large may capture multiple objects in the scene, thus making it difficult to differentiate and identify objects in the scene. Conversely, however, if the pixel size is too small, the intensity of electromagnetic radiation from some objects in the scene that is captured by each pixel will be lower, possibly decreasing the signal-to-noise ratio for any given pixel, thus resulting in unreliable and hard-to-read hyperspectral images.

### BRIEF SUMMARY OF THE INVENTION

It is an objective of the present invention to provide systems and methods that allow for hyperspectral medical imaging. Embodiments of the invention are provided herein. Embodiments of the present invention can be freely combined with each other if they are not mutually exclusive.

The present invention may be used to image in vivo or ex vivo tissue. Without wishing to limit the present invention, the present invention has particular utility for imaging tumors and cancerous tissues and distinguishing their spectral signatures from nearby healthy/non-diseased tissue. For example, the present invention may be used intraoperatively to image an excised ex vivo tissue sample removed during a mastectomy or a lumpectomy. Due to the quick turnaround time of the image processing capabilities of the present invention, a surgical team performing the mastectomy or lumpectomy may examine the hyperspectral images produced using the present invention to determine if the ex vivo tissue was removed with adequate surgical margins. If the margins of the excised ex vivo tissue are inadequate, the surgical team can then adjust their surgical technique accordingly.

Alternatively, the present invention may be used intraoperatively to image in vivo tissue. For example, the surgical team during the mastectomy or lumpectomy could instead use the present invention to produce a hyperspectral image of the surgical field or resection pocket after excision of the tumor. If the margins seen in the hyperspectral image of the surgical field or resection pocket are inadequate (i.e. to demonstrate residual tumor left in the patient), the surgical team can similarly adjust their surgical technique accordingly.

In yet another potential use of the present invention, the surgical team may use the present invention to produce a hyperspectral image of the surgical site before making an incision, or after making an incision to expose tissue and prior to making a resection or removing tissue. They may thus make surgical decisions accordingly, with greater knowledge of the size, location, and extent of the tumor to be excised. In vivo use of the present invention is particularly advantageous because the present invention does not require the use of dyes, radiocontrast agents, or other agents introduced into the patient's body. Many of these agents have undesirable toxicities. Additionally, the process of obtaining an image is made simpler and easier by the present invention, because it does not require additional steps associated with the use of these agents. The present invention's ability to produce high-quality images without the use of these agents is thus advantageous and is a result of the unique performance characteristics of the system with respect to the combination of spectral range, spectral resolution, and spatial resolution.

The present invention features a hyperspectral medical imaging system for imaging a tissue. The hyperspectral medical imaging system may comprise a light source configured to emit incident light. The incident light may reflect off the tissue as reflected light, pass through the tissue as transmitted light, or a combination thereof. The system may further comprise an adjustable transmissive optical component configured to transmit up to all portions of the reflected light as needed to achieve the desired imaging characteristics. The system may further comprise a hyperspectral sensor configured to detect a property of the reflected light, the transmitted light, or the combination thereof transmitted through the transmissive optical component. The hyperspectral sensor may be further configured to capture a raw hyperspectral image and transmit the raw hyperspectral image to a computing device. The system may further comprise the computing device operatively connected to the hyperspectral sensor. The computing device may comprise a processor capable of executing computer-readable instructions and a memory component comprising the computer-readable instructions. The computing device may be configured to process the raw hyperspectral image to produce a processed hyperspectral image, and transfer the processed hyperspectral image to the memory component. The system may further comprise a display operatively connected to the computing device. The computing device may be further configured to read the processed hyperspectral image from the memory component and display the processed hyperspectral image on the display.

The processed hyperspectral image may be displayed on the display in a manner that is manipulable by an operator. The incident light may be from about 400 nm to about 2500 nm or a subset or combination of subsets within this range according to the application. In some embodiments, the raw hyperspectral image may be processed in real-time such that the color-coded image displayed on the display is displayed and updated in five seconds or less. The color-coded image may be projected onto the subject, whether it is the in-vivo or ex-vivo tissue, to display the color-coded image onto the subject.

The present invention features a method of producing a hyperspectral image. The method may comprise illuminating an in vivo or ex vivo tissue specimen with a light source. The method may further comprise obtaining a scout image of the tissue specimen using a hyperspectral sensor to increase a hyperspectral image quality. The method may further comprise obtaining the hyperspectral image by capturing a reflectance spectrum, a transmission spectrum, or a combination thereof of one or more faces the tissue specimen using the hyperspectral sensor. The method may further comprise saving the hyperspectral image as a datafile to a computing device, the computing device comprising a processor capable of executing computer-readable instructions and a memory component comprising computer-readable instructions for processing the hyperspectral image obtained by the hyperspectral sensor.

The method further comprises extracting a set of endmember spectra from the image. The method may further comprise saving spectra to a spectral library. The method may further comprise methods for manipulating a set of spectra compiled into a library. The method may further comprise using one or more spectra from the library or extracted from the image as endmembers for unmixing. A set of endmembers may represent the set of tissue types present in the field of view. In some embodiments, the method may further comprise unmixing the image using endmembers extracted from the image or read from a spectral library. In other embodiments, the method may further comprise unmixing the image by using the endmembers to express each pixel in the image as a linear or nonlinear combination of endmembers. The method may further comprise using the computing device to generate a color-coded image from an unmixed hyperspectral image. The color-coded image may further delineate the boundary between regions in the image. The method may further comprise displaying the color-coded image on a display operatively connected to the computing device or projected onto the subject. The color-coded image may contain one or more pixels whose color is determined by the combination of endmembers determined by the unmixing process. The combination may represent the relative abundance of each endmember's tissue type.

In some embodiments, obtaining an endmember from the hyperspectral image further comprises obtaining an endmember as the spectrum captured by at least one sensor pixel. In some embodiments, obtaining an endmember from the hyperspectral image further comprises obtaining an endmember from at least one component of the hyperspectral image. In some embodiments, the endmember may be obtained directly from the hyperspectral image or the endmember may be retrieved from a library of endmember data. In some embodiments, the endmember represents in the wavelength bands a material in the field of view used to collect the hyperspectral image. In some embodiments, the endmember represents spectral features of a material. In some embodiments, the endmember represents a spectral profile of a material. In some embodiments, the endmember represents a spectral signature of a material.

The method may further comprise selecting at least one endmember. Selecting may comprise randomly or pseudo-randomly selecting a number of at least one pixel to represent a set of at least one endmember, each of the at least one pixel representing a pixel cluster, the pixel cluster comprising pixels nearest to one of the at least one endmembers as compared to other endmembers. Selecting may further comprise assigning each of the at least one pixel with a cluster identifier corresponding to the pixel cluster the at least one pixel is a part of. Selecting may further comprise labeling each pixel of the pixel cluster with its nearest endmember. Selecting may further comprise repeating assigning and labeling for all pixels selected randomly or pseudo-randomly. Selecting may further comprise computing a mean spectrum for the pixel cluster. Selecting may further comprise calculating a mean cluster change for the pixel cluster. The mean cluster change may be a change between a current mean spectrum of the pixel cluster and a previous mean spectrum of the pixel cluster. Selecting may further comprise finding a maximum mean cluster change among the calculated mean cluster changes. Selecting may further comprise repeating computing, calculating, and finding the maximum mean cluster change until the maximum mean cluster change is at or below a threshold value. Selecting may further comprise utilizing each mean spectrum as an endmember for unmixing or adding to a spectral library. The endmember may be a reference candidate spectrum and the endmember represents in the wavelength bands a material in the field of view used to collect the hyperspectral image.

Unmixing the hyperspectral image may comprise obtaining a pixel spectrum recorded by at least one sensor pixel in the hyperspectral sensor, the at least one sensor pixel corresponding to an image pixel in the hyperspectral image. The pixel spectrum may represent a reflectance spectrum, a transmission spectrum, or a combination thereof in a field of view of the at least one sensor pixel. Unmixing may further comprise comparing the pixel spectrum to at least one endmember. A linear or non-linear combination of the at least one endmember representing the pixel spectrum is then found. Each endmember coefficient in that combination may represent the relative proportion of the tissue types present in the sample in the pixel's field of view. Each of the endmember coefficients may thus correspond to a proportion of tissue type present in the tissue specimen location in or on the tissue specimen. Each image pixel in the hyperspectral image may assume an image pixel color on the display corresponding to a relative proportion of each endmember and/or tissue type present in the tissue specimen location in or on the tissue specimen.

The method may further comprise principal component analysis. The method may further comprise performing principal components analysis on a hyperspectral image, selecting the first few principal components, which are spectra with the same number of wavelengths as each pixel, using those components as endmembers, and unmixing using those components. The method may further comprise adding those components to a spectral library.

The method may further comprise obtaining a reference tissue comprising at least one known tissue type or disease state. The method may further comprise obtaining a reference pixel spectrum using the hyperspectral sensor for a point on the reference tissue. The method may further comprise storing the reference pixel spectrum, or extracted features of said spectrum, corresponding to the reference tissue in the endmember library. The method may further comprise obtaining a clinical pixel spectrum using the hyperspectral sensor for a point on an in vivo or ex vivo clinical tissue. The method may further comprise determining at least one clinical tissue type present in the clinical tissue by comparing the clinical pixel spectrum to the reference pixel spectrum. The at least one clinical tissue type may be determined to be the at least one tissue type corresponding to the reference tissue with the reference pixel spectrum most similar to the clinical pixel spectrum. The relative abundance of clinical tissues present in a pixel's field of view may be determined as the coefficients in the combination of endmembers representing the pixel's recorded spectrum. The method of producing the hyperspectral image may be performed in real-time such that the color-coded image displayed on the display is displayed and updated in five seconds or less.

The method may further comprise obtaining a tissue sample, obtaining at least one region-of-interest (ROI) pixel spectrum using the hyperspectral sensor for at least one user-selected ROI on the tissue sample, storing the ROI pixel spectrum corresponding to the tissue sample in the endmember library as at least one ROI endmember, and using the computing device to unmix the hyperspectral image against the ROI endmember. The at least one ROI endmember may define a tissue type of the at least one ROI. The ROI may comprise a point, a region, or a combination thereof.

The present invention features a hyperspectral medical imaging system for imaging a tissue. The system may comprise a light source emitting broadband light from about 400 nm to about 2500 nm, the light source configured to emit incident broadband light, the incident broadband light reflecting off the tissue as reflected light, passing through the tissue as transmitted light, or a combination thereof. The system may further comprise a polarizer configured to transmit the reflected light through a lens. The system may further comprise the lens configured to focus and transmit the reflected light, the transmitted light, or the combination thereof through a spectrograph to a hyperspectral sensor. The system may further comprise the hyperspectral sensor configured to detect a property of the reflected light, the transmitted light, or the combination thereof, the hyperspectral sensor further configured to capture a raw hyperspectral image and transmit the raw hyperspectral image to a computing device. The system may further comprise the computing device operatively connected to the hyperspectral sensor. The computing device may comprise a processor capable of executing computer-readable instructions and a memory component comprising computer-readable instructions. The computing device may be configured to process the raw hyperspectral image to produce a processed hyperspectral image and transfer the processed hyperspectral image to the memory component. The system may further comprise a display operatively connected to the computing device. The computing device may be further configured to read the processed hyperspectral image from the memory component and display the processed hyperspectral image on the display. The system may further comprise a spectrograph disposed optically in-line with the transmissive optical component and the hyperspectral sensor, the spectrograph comprising an opening configured to accept the reflected light, the transmitted light, or the combination thereof, and a dispersive element configured to spread a spectra of the reflected light, the transmitted light, or the combination thereof into a plurality of spectral bands focused towards the hyperspectral sensor.

One of the unique and inventive technical features of the present invention is the ability to quickly obtain an image of tissue and produce an estimate of the identities of materials present in the field of view. This is useful in a number of settings, but is particularly useful intraoperatively, especially in tumor resection. Without wishing to limit the invention to any theory or mechanism, it is believed that the technical feature of the present invention advantageously provides for improved clinical outcomes, especially in patients undergoing tumor resection. None of the presently known prior references or work has the unique inventive technical feature of the present invention.

For example, cryosection (i.e., frozen section biopsy or frozen section procedure) is the current standard of care for certain types of tumor margin assessment. This technique involves flash freezing of the biopsied tissue followed by a “rapid stain procedure.” Compared to traditional tissue staining methods, which may take 24 hours or more, cryosection trades accuracy for rapidity. Cryosectioning still takes roughly 20 minutes to perform in a lumpectomy. Cryosection is associated with sensitivity values of between 60% to 90%, and specificity values of 65% to 90%. However, this varies between pathologists (as cryosection is heavily dependent upon the skill of a given pathologist performing the cryosection and interpretation) and also the type of tissue involved (as, for example, breast tissue has a different response to the cryosection protocol than liver tissue does). Comparatively, the present invention can capture and process an image in roughly one minute and has demonstrated sensitivity and specificity of 90% or more in trials thus far.

Cryosectioning is also limited in that it only analyzes a comparatively small portion of the biopsied tissue intraoperatively. For example, if a 5×5×5 cubic inch specimen were obtained, intraoperatively cryosectioning would only examine perhaps 0.5×0.5 square inch samples from each face of the biopsied tissue for the sake of achieving a rapid result, with analysis of the full 5×5 square inch face performed after the surgery has ended. If the tumor was not captured in the sampled area, this can lead to a false negative. Such false negatives are common, and result in patients being informed days after the surgery is completed (when the “full” staining results are obtained) that some cancerous tissue was “left behind.” This results in increased healthcare costs, and increased morbidity and mortality to patients. Comparatively, the present invention is capable of producing a hyperspectral image of the entirety of each face of the biopsied tissue, greatly reducing the chance of false positives that lead to the necessity of subsequent surgery. Other existing techniques used for intraoperative surgical margin assessment include clinical radiography and optical coherence tomography.

Clinical radiography suffers from the shortcoming that it is useful for imaging the tissue sample containing the excised tumor, but not the in vivo tissue picket. The excised tissue containing the tumor is placed in a shielded X-ray cabinet. Multiple X-ray images of the resected tissue, from different perspectives, are obtained. X-ray images are used to observe surrogate markers for cancer present in the tissue. For example, some types of advanced breast tumors have microcalcifications scattered throughout. If present at the edges of the resected tissue, this suggests that not all of the tumor has been removed. However, this approach does not work for many tumor types because they do not contain microcalcifications or other surrogate markers detectable by clinical radiography. Also, surrogate markers like microcalcifications only offer a rough estimate of a tumor's true margin. Third, in patients who have received prior chemotherapy and/or radiation treatment, the microcalcifications remain despite the tumor itself shrinking, leading to false positives. Fourth, because this method relies on the presence of microcalcifications, it is limited to the analysis of breast tissue, and even then only to breast tissue that has been excised. Comparatively, the present invention can indicate that a given region is tumor-free without excision from the patient by imaging the surgical field or resection pocket. The present invention also has potential utility far beyond breast cancer detection because it does not rely on the presence of microcalcifications.

Optical coherence tomography (OCT) is another technique used to assess tumor margins on resected samples. It also suffers from a number of shortcomings. First, it may only be used to examine the margins of removed specimens, and thus may not be used to examine the surgical pocket or resection pocket, nor examine the location, size, and extent of the tumor before excision. Second, OCT is based on displaying high-resolution images of cellular architecture and relies on expert interpretation by the surgeon in the room, which may be prone to variation between surgeons and/or human error. Third, while OCT does achieve a high resolution, it does so via a focused and very narrow field of view, which results in a time-intensive scanning procedure. For example, it typically takes 12 minutes to scan a 2 inch×2 inch sample of tissue with OCT. Fourth, OCT works best for well-organized tissues, such as the retina, which always consists of the same tissue layers. Using OCT for disorganized tissues poses significant challenges, as the images become very difficult to interpret, and require extensive special training to determine which changes in cellular morphology are associated with each type and stage of cancer. Interpretation of OCT data is thus far from trivial and is associated with numerous interpretation problems. These problems are so well-known, in fact, that current efforts are underway to use artificial intelligence (Al) to interpret OCT images in cancer margins, thus mitigating these shortcomings. Comparatively, the present invention may be used on both in vivo and ex vivo specimens, requires far less training to interpret, achieves faster scan times, works better for disorganized tissues, and is not associated with the same degree of difficulty in interpretation and the same likelihood of human error in interpretation.

Thus, the present invention offers more accurate results than existing methods, which are achieved faster, and which are more intuitive and require less training to interpret. The present invention produces an image that, for example, easily allows the surgical team to identify the size, location, and extent of a tumor, or easily visualize a tumor's margins. Furthermore, the present invention is non-contact, does not require the use of dyes or other chemical agents, may be performed at the point of care by the surgical team, and requires little to no training to interpret, as it produces a color-coded image.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and embodiments.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a hardware schematic of an embodiment of the present invention.

FIG. 2 shows a software algorithm of an embodiment of the present invention.

FIG. 3 shows a pseudocolor image of an excised breast tissue sample produced using systems and methods of the present invention, containing healthy tissue (black), normal fat, tumor, and background.

FIG. 4 shows a composite image containing four images of a single excised breast tissue sample. The first image is a hyperspectral image showing normal tissue in white. The second image is a hyperspectral image showing normal fat in white. The third image is a hyperspectral image showing cancer in white. The final image is a hematoxylin and eosin (H&E) pathology stain, with a line indicating the border between healthy tissue (in the upper portion of the sample) and cancer (in the lower portion of the sample). This may be compared to the hyperspectral images in the first three images, which indicate the same line of demarcation (plus additional data) without the need for time-and labor-intensive staining and expert interpretation by a pathologist.

### DETAILED DESCRIPTION OF THE INVENTION

The term “endmember” is defined herein as a reflectance or transmission spectrum used to represent a material in a hyperspectral image. For instance, a spectrum may be identified as a typical reflectance spectrum for cancerous tissue. It does not need to be a recorded spectrum; it may be an artificially constructed spectrum.

The term “hyperspectral image” is defined herein as a three-dimensional array of reflectance data, typically decomposed as a two-dimensional rectangular array of pixels, each of which is a one-dimensional vector called a pixel spectrum.

The term “pixel spectrum” is defined herein as a one-dimensional reflectance spectrum recorded in at least one pixel by a hyperspectral camera.

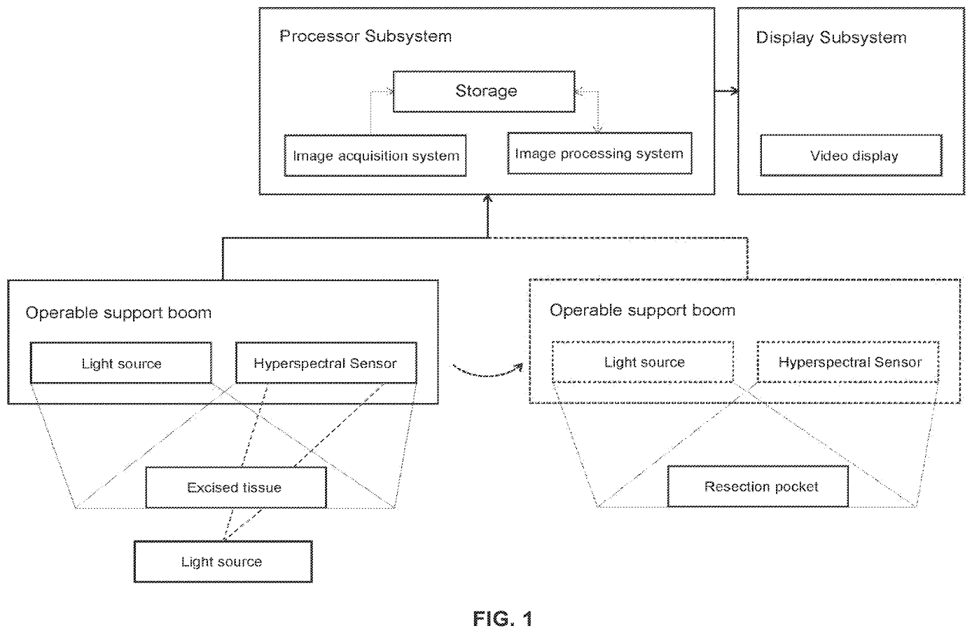

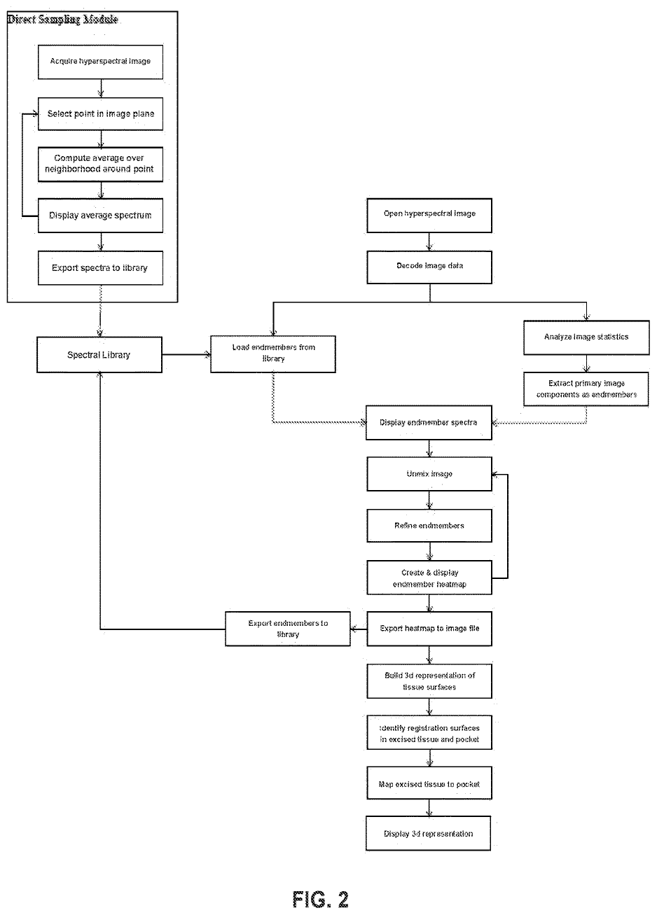

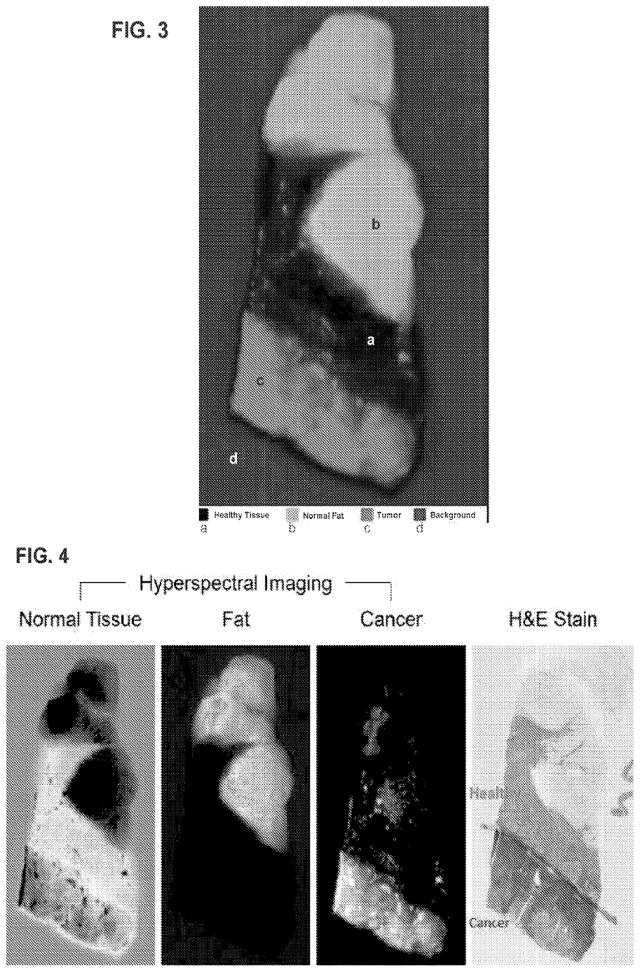

Referring now to FIGS. 1-4, the present invention features a hyperspectral medical imaging system for imaging a tissue. The hyperspectral medical imaging system may comprise a light source configured to emit incident light. The incident light may reflect off the tissue as reflected light. The incident light may be from about 400 nm to about 2500 nm. It may include only certain subsets or combinations of subsets of light within that range. Alternatively, the light may be placed behind the tissue specimen in order to measure the transmitted rather than reflected spectrum. The system may further comprise a transmissive optical component configured to transmit the reflected or transmitted light. In some embodiments, the transmissive optical component may comprise an adjustable transmissive optical component configured to transmit up to all portions of the reflected light as needed to achieve the desired imaging characteristics with respect to brightness, glare, and contrast. In some embodiments, the adjustable transmissive optical component may comprise a polarizer. In some embodiments, the polarizer may comprise a neutral density filter. In some embodiments the transmissive optical component may comprise a liquid crystal tunable filter (LCFT) or an acousto-optic tunable filter (AOTF). In some embodiments the transmissive optical component may comprise a narrow slit or series of slits. In some embodiments the transmissive optical component may comprise diffraction elements, prisms, and/or collimators.

The system may further comprise a hyperspectral sensor configured to detect a property of the reflected or transmitted light passing through the transmissive optical component or components. The hyperspectral sensor may be further configured to capture a raw hyperspectral image and transmit the raw hyperspectral image to a computing device. The system may further comprise the computing device operatively connected to the hyperspectral sensor. The computing device may comprise a processor capable of executing computer-readable instructions and a memory component comprising the computer-readable instructions. The computing device may be configured to process the raw hyperspectral image to produce a processed hyperspectral image, and transfer the processed hyperspectral image to the memory component. The system may further comprise a display operatively connected to the computing device. The display may further comprise elements that allow users to display spectra, regions of the image, or zoom, rotate, or otherwise manipulate and annotate the image as desired. The computing device may be further configured to read the processed hyperspectral image from the memory component and display the processed hyperspectral image on the display. The processed hyperspectral image may be displayed on the display and is manipulable by an operator. The raw hyperspectral image may be processed in real-time such that the color-coded image displayed on the display is displayed and updated in five seconds or less.

The present invention features a method of producing a hyperspectral image. The method may comprise illuminating an in vivo or ex vivo tissue specimen with a light source. The method may further comprise obtaining a scout image of the tissue specimen using a hyperspectral sensor and subsequently adjusting image acquisition parameters to increase a hyperspectral image quality. The method may further comprise obtaining the hyperspectral image by capturing a reflectance spectrum of a face of the tissue specimen using the hyperspectral sensor or by capturing the spectrum of light transmitted through a sufficiently thin and uniform specimen in the case of transmission spectrum imaging. The method may further comprise saving the hyperspectral image as a datafile to a computing device, the computing device comprising a processor capable of executing computer-readable instructions and a memory component comprising computer-readable instructions for processing the hyperspectral image obtained by the hyperspectral sensor.

The method may further comprise obtaining at least one endmember from at least one of the hyperspectral images or an endmember library. The at least one endmember may be a reference candidate spectrum. The at least one endmember may represent a material in wavelength bands used to collect the hyperspectral image. The method may further comprise using the computing device to process the datafile to unmix the hyperspectral image against the at least one endmember.

Unmixing the image may comprise defining a distance measure on the space of pixel spectra, such as the squared Euclidean distance, and identifying a set of endmembers as described elsewhere (library, ROI-based, PCA, etc.). For each pixel in the image, unmixing may further comprise obtaining a linear or nonlinear combination of endmembers which minimizes the distance between the combination and the pixel. The coefficients of the combination are the abundances of the endmembers in the pixel spectrum. In some embodiments, unmixing does not happen sequentially; the present method unmixes with a set of endmembers concurrently. Once a given number of endmembers are obtained, then the unmixing happens. In other embodiments, unmixing happens sequentially and the image may be unmixed against each received endmember one at a time. The method may further comprise using the computing device to generate a color-coded image from an unmixed hyperspectral image. The method may further comprise displaying the color-coded image on a display operatively connected to the computing device. The color-coded image may contain a color-coded pixel, the color-coded pixel corresponding to the proportions of endmembers which best represent the pixel spectrum. Each endmember may correspond to a distinct tissue/material type within the image.

Unmixing the hyperspectral image may comprise obtaining a pixel spectrum recorded by at least one sensor pixel in the hyperspectral sensor which corresponds to an image pixel of a plurality of image pixels in the hyperspectral image. In some embodiments, recording the at least one sensor pixel may comprise implementing a line scan system. The line scan system may record an image in 1 pixel wide by “X” pixel long sections (i.e. a line) with the complete spectral profile of the material captured for every individual pixel along the line. The dispersive element of the spectrograph arranges each band of light into adjacent bands, and each is measured by a separate spot in the sensor. In other words, the spectrum of each individual pixel is split apart and each individual wavelength (900 nm, 905 nm, 910 nm, etc.) is focused on a different spot of the detector. The plurality of bands represent the complete spectral profile of that point in space spread across the detector, for each image pixel down the line. Then, the scanner advances one line and the next is recorded. There is an imaging platform that slides beneath the imager so that a full image can be produced by getting line after line. In other embodiments, the camera or its direction of focus can be shifted to effectively scan each subsequent line.

The pixel spectrum may represent a reflectance spectrum, a transmission spectrum, or a combination thereof in a field of view of the at least one sensor pixel. Unmixing may further comprise comparing the pixel spectrum to all endmembers. Unmixing may further comprise estimating a composition of materials in the field of view of the at least one sensor pixel by comparing the pixel spectrum corresponding to the at least one sensor pixel to the at least one endmember and constructing at least one combination of at least one endmember that approximates the pixel spectrum recorded by the at least one sensor pixel represented as the image pixel in the hyperspectral image.

Unmixing may further comprise expressing each image pixel in the hyperspectral image as a combination of at least one endmember. Each endmember of the combination of the at least one endmember represented in each image pixel of the hyperspectral image may be associated with an endmember coefficient. Each endmember coefficient may represent a proportion of the tissue specimen that corresponds to at least one endmember present in the tissue specimen location in or on the tissue specimen. Each of the endmember coefficients may thus correspond to a proportion of tissue type present in the tissue specimen location in or on the tissue specimen. Each image pixel in the hyperspectral image may assume an image pixel color on the display corresponding to a relative proportion of each endmember and tissue type present in the tissue specimen location in or on the tissue specimen.

The method may further comprise selecting at least one endmember. Selecting may comprise a k-means clustering process. The process may comprise selecting a number of pixels. In some embodiments, the number of pixels may be randomly selected, non-randomly selected, or a combination thereof. The process may further comprise computing, for each pixel of the number of pixels, the nearest vector. The process may further comprise labeling each pixel of the number of pixels with its corresponding nearest vector. The process may further comprise computing, for each label, the average vector for all pixels with that label. The process may further comprise repeating computing the nearest vector, labeling each pixel, and computing the average vector until the maximum change becomes sufficiently small.

The mean cluster change may be a change between a current vector of the pixel cluster and a previous vector of the pixel cluster. Selecting may further comprise finding a maximum mean cluster change among the calculated mean cluster changes. Selecting may further comprise repeating computing, calculating, and finding the maximum mean cluster change until the maximum mean cluster change is at or below a threshold value. Selecting may further comprise utilizing each vector as an endmember for unmixing. The endmember may be a reference candidate spectrum and the endmember represents in the wavelength bands a material in the field of view used to collect the hyperspectral image. In some embodiments, the calculated mean may be added to a library.

The method may further comprise obtaining a reference tissue comprising a known tissue type and/or disease state of said tissue. The method may further comprise obtaining a reference pixel spectrum using the hyperspectral sensor for a point on the reference tissue. The method may further comprise storing the reference pixel spectrum corresponding to the reference tissue in the endmember library. The method may further comprise obtaining a clinical pixel spectrum using the hyperspectral sensor for a point on an in vivo or ex vivo clinical tissue. The method may further comprise determining a clinical tissue type present in the clinical tissue by comparing the clinical pixel spectrum to the reference pixel spectrum. In some embodiments, the reference pixel spectrum may be derived from a library. The clinical tissue type may be determined to be the known tissue type corresponding to the reference tissue with the reference pixel spectrum most similar to the clinical pixel spectrum. The method of producing the hyperspectral image may be performed in real-time such that the color-coded image displayed on the display is displayed and updated in five seconds or less. In some embodiments, principal component analysis may be performed to determine the endmembers. In some embodiments, the endmember library may be fully defined by spectral data based on collected tissue samples having known tissue types and diseases.

The present invention features a hyperspectral medical imaging system for imaging a tissue. The system may comprise a light source emitting broadband light from about 400 nm to about 2500 nm, the light source configured to emit incident broadband light, the incident broadband light reflecting off the tissue as reflected light. The light source may alternatively be configured such that emitted light is transmitted through sufficiently thin samples to record a transmission spectrum. The system may further comprise the polarizer configured to filter and transmit the reflected or transmitted light. The system may further comprise a lens configured to focus and transmit the reflected or transmitted light. Additional optical elements such as objective lenses and neutral density filters may be used to adjust the focal depth, field of view, working distance, and magnification as best suited to the particular application.

In some embodiments, the lens may be configured to focus and transmit the reflected or transmitted light to a spectrograph. The spectrograph may comprise an opening and dispersive elements positioned past the opening. The dispersive elements may be configured to spread out the spectra from a single line into a plurality of spectral bands. The narrower the opening, the more spectral bands are generated. This is then focused onto the sensor such that every row of the sensor contains intensity information of a different wavelength.

The system may further comprise the hyperspectral sensor configured to detect a property of the reflected or transmitted light, the hyperspectral sensor further configured to capture a raw hyperspectral image and transmit the raw hyperspectral image to a computing device. The system may further comprise a movable support boom operatively coupled to the optical components (i.e. lenses), light source(s), spectrograph and hyperspectral sensor such that the assembled components can be moved as one unit and optimally positioned with respect to the desired field of view. The system may further comprise the computing device operatively connected to the hyperspectral sensor. The computing device may comprise a processor capable of executing computer-readable instructions and a memory component comprising computer-readable instructions. The computing device may be configured to process the raw hyperspectral image to produce a processed hyperspectral image, and transfer the processed hyperspectral image to the memory component. The system may further comprise a display operatively connected to the computing device the display and the hyperspectral sensor. The computing device may be further configured to read the processed hyperspectral image from the memory component and display the processed hyperspectral image on the display.

The present invention features a hyperspectral medical imaging system for imaging a tissue. In some embodiments, the hyperspectral medical imaging system may comprise a hyperspectral sensor. The system may further comprise transmissive optical components. The system may further comprise a light source. The system may further comprise a display. The system may further comprise a computing device. The computing device may be operatively connected to the display and the hyperspectral sensor. The computing device may comprise a processor capable of executing computer-readable instructions and a memory component comprising computer-readable instructions. The system may further comprise a housing with an internal coating and finish selected to minimize scatter and glare selected to optimize imaging performance. The system may further comprise an imaging stage with a known spectral profile to allow for calibration and to minimize scatter and glare. The system may further comprise adjustable lighting elements to allow for customizable illumination of the target field of view.

The light source may be configured to emit incident light, the incident light reflecting off the tissue as reflected light. The system may alternatively be configured such that the emitted light passes through the specimen. The transmissive optical component may be configured to transmit the reflected or transmitted light (i.e. the light coming from the sample) to the spectrograph/sensor. The hyperspectral sensor may be configured to detect a property of the reflected (or transmitted) light transmitted through the transmissive optical component. The hyperspectral sensor may be further configured to capture the raw hyperspectral image and transmit the raw hyperspectral image to the computing device.

The computing device may be configured to process the raw hyperspectral image to produce a processed hyperspectral image. The computing device may be configured to transfer the processed hyperspectral image to the memory component. The computing device may be further configured to read the processed hyperspectral image from the memory component and display the processed hyperspectral image on the display. In some embodiments, the display may comprise a computer monitor, a television, a projector, or other device capable of displaying an image. In some embodiments, computer-readable instructions may comprise processing the raw hyperspectral image and reading the processed hyperspectral image. In some embodiments, the displayed image will be modifiable by the user, allowing for selection of specific spectral profiles, pseudo coloration, annotation, storing images, selection of regions of interest, zooming, rotating, and other changes.

In some embodiments, the present invention features a method of producing a hyperspectral medical image. The method may comprise illuminating an in vivo or ex vivo tissue specimen with a light source. The method may further comprise obtaining a scout image of the tissue specimen using a hyperspectral sensor to increase hyperspectral image quality. The method may further comprise obtaining the hyperspectral image by capturing a reflectance spectrum of a face of the tissue specimen using the hyperspectral sensor. The method may further comprise obtaining a hyperspectral image by measuring the transmission spectrum of light passing through the tissue using the hyperspectral sensor. The method may further comprise saving the hyperspectral image as a datafile.

The method may further comprise transferring the datafile comprising the hyperspectral image to a computing device. The computing device may comprise a processor capable of executing computer-readable instructions and a memory component comprising computer-readable instructions for processing the hyperspectral image obtained by the hyperspectral sensor. The method may further comprise obtaining an endmember from at least one of the hyperspectral images or an endmember library. The endmember may be a reference candidate spectrum. The endmember may represent in the wavelength bands a material in the field of view.

The method may further comprise using the computing device to process the datafile to unmix the hyperspectral image against the endmember. The method may further comprise using the computing device to generate a color-coded image from an unmixed hyperspectral image. The method may further comprise displaying the color-coded image on a display. The color-coded image may contain a colored pixel, the colored pixel corresponding to at least one endmember, each endmember corresponding to a tissue type present in a tissue specimen location in or on the tissue specimen.

In some embodiments, obtaining an endmember from the hyperspectral image may comprise obtaining an endmember from the spectrum captured by at least one sensor pixel. In some embodiments, obtaining an endmember from the hyperspectral image may further comprise obtaining an endmember from at least one portion of the hyperspectral image. In some embodiments, the endmember may be obtained directly from the hyperspectral image or the endmember may be retrieved from a library of endmember data.

In some embodiments, the endmember may represent in the wavelength bands a material in the field of view used to collect the hyperspectral image. In some embodiments, the endmember may represent the spectral feature(s) of a material. In some embodiments, the endmember may represent a spectral profile of a material. In some embodiments, the endmember may represent a spectral signature of a material.

In some embodiments, unmixing the hyperspectral image may comprise obtaining a pixel spectrum recorded by at least one sensor pixel in the hyperspectral sensor which corresponds to an image pixel in the hyperspectral image. The pixel spectrum may represent a reflectance or transmission spectrum in the field of view of the at least one sensor pixel. Unmixing may further comprise comparing the pixel spectrum to at least one endmember. Unmixing may further comprise estimating the composition of a material in the field of view of the at least one sensor pixel by comparing the pixel spectrum corresponding to the at least one sensor pixel to the at least one endmember and constructing a combination of at least one endmember that approximates the pixel spectrum recorded by the at least one sensor pixel represented as the image pixel in the hyperspectral image. Unmixing may further comprise expressing each image pixel in the hyperspectral image as a combination of at least one endmember.

In unmixing an image, each pixel may be represented as a linear or non-linear combination of at least one endmember. Each endmember coefficient represents the relative degree to which the image pixel corresponds to the at least one endmember. Each image pixel in the hyperspectral image may assume an image pixel color on the display corresponding to the relative proportion of each endmember present in the tissue specimen location in or on the tissue specimen. In some embodiments, unmixing the hyperspectral image may comprise estimating the composition of a material in the field of view of the at least one sensor pixel. In some embodiments, unmixing the hyperspectral image may comprise estimating the abundance of a material in the field of view of the at least one sensor pixel. In some embodiments, the colored pixel may correspond to an endmember. In some embodiments, the colored pixel may correspond to the relative abundance of endmembers present in the pixel's field of view.

In some embodiments, the degree of matching between a combination of endmembers and an observed pixel spectrum may be determined using a least-means-squares approach, including a least-means-squares approach that determines the linear combination of endmembers that minimizes the distance between the linear combination of endmembers and the recorded pixel spectrum; distance is measured in the vector space whose number of dimensions is the number of spectral bands in the hyperspectral image. As a non-limiting example, in some embodiments, such an approach may be used to compare the pixel spectrum to a set of endmembers and estimate the composition of a material in the field of view of the at least one sensor pixel by comparing the pixel spectrum corresponding to the at least one sensor pixel to the set of endmembers and constructing a combination of at least one endmember that approximates the pixel spectrum.

In some embodiments, unmixing may comprise a k-means algorithm. In some embodiments, the method may further comprise selecting at least one endmember. In some embodiments, selecting may comprise randomly or pseudo-randomly selecting a number (k) of at least one pixel (p) to represent a set of at least one endmembers, each of the at least one pixel representing a pixel cluster, the pixel cluster comprising pixels nearest to one of the at least one endmembers as compared to other endmembers. Selecting may further comprise assigning each of the at least one pixel with a cluster identifier corresponding to the pixel cluster the at least one pixel is a part of. Determining which pixel cluster the at least one pixel is a part of may comprise finding the closest cluster mean. This may be used to determine the mean nearest to each pixel of the pixel cluster. Selecting may further comprise labeling each pixel of the pixel cluster with its nearest mean. Selecting may further comprise repeating the aforementioned steps for all pixels selected. Selecting may further comprise computing a mean spectrum for a pixel cluster. Selecting may further comprise calculating a mean cluster change for the pixel cluster. The mean cluster change may be the change between the magnitude of the current mean spectrum of the pixel cluster and the magnitude of the previous mean spectrum of the same pixel cluster. Selecting may further comprise finding a maximum mean cluster change among the calculated mean cluster changes. Selecting may further comprise repeating the aforementioned steps until the maximum mean cluster change is at or below a threshold value. Selecting may further comprise utilizing each resulting mean spectrum as an endmember for unmixing. The endmember may be a reference candidate spectrum and the endmember represents in the wavelength bands a material in the field of view used to collect the hyperspectral image. In some embodiments, the endmember(s) may be added to a library. the change or distance, Euclidean or otherwise, between the cluster's mean spectrum and its previous mean.

In some embodiments, the method may further comprise obtaining a reference tissue comprising a known tissue type. The method may further comprise obtaining a reference pixel spectrum using a hyperspectral sensor for a point on the reference tissue. The method may further comprise obtaining a reference pixel spectrum based on a collection of points of the same known tissue type from the reference tissue. The method may further comprise storing the reference pixel spectrum corresponding to the reference tissue in an endmember library. The method may further comprise obtaining a clinical pixel spectrum using a hyperspectral sensor for a point on an in vivo or ex vivo clinical tissue. The method may further comprise estimating abundances of clinical tissue types by comparing the clinical pixel spectrum to the reference pixel spectrum.

In some embodiments, determining a reference pixel spectrum comprises using pathology results to validate the selection of endmembers from regions of established tissue type or disease state. These endmembers may be stored as reference pixel spectra. Hyperspectral images of pathology validated tissue types can be unmixed using these endmembers which enables verification of endmember spectra. In some embodiments, the degree of similarity deemed sufficiently similar may be determined a priori by a user of the systems or methods of the present invention. In some embodiments, determining a clinical tissue type present in the clinical tissue comprises comparing the clinical pixel spectrum to the reference pixel spectrum.

In some embodiments, the present invention features a hyperspectral medical imaging system for imaging a tissue. The system may further comprise a polarizer. The system may further comprise a lens or series of lenses, prisms, diffraction gratings, filters; both tunable and non-tunable, slits, mirrors, and interferometric components. The hyperspectral medical imaging system may comprise a hyperspectral sensor. The system may further comprise a light source emitting broadband light from about 400 nm to about 2500 nm. The light source or sources may further be moveable with respect to the field of view. The system may further comprise a movable support boom. The optical elements, spectrograph, and hyperspectral sensor may be operatively coupled to the moveable support boom. The system may further comprise a display. The system may further comprise an imaging stage or platform with predefined spectral characteristics. The system may further comprise a computing device. The computing device may be operatively connected to the display and the hyperspectral sensor. The computing device may comprise a processor capable of executing computer-readable instructions and a memory component comprising computer-readable instructions.

The light source may be configured to emit incident broadband light. The incident broadband light may reflect off the tissue as reflected light. The polarizer and the lens may be configured to transmit the reflected light such that the reflected light strikes the hyperspectral sensor. The hyperspectral sensor may be configured to detect a property of the reflected light. The hyperspectral sensor may be further configured to capture a raw hyperspectral image and transmit the raw hyperspectral image to the computing device. The computing device may be configured to process the raw hyperspectral image to produce a processed hyperspectral image. The computing device may be further configured to transfer the processed hyperspectral image to the memory component. The computing device may be further configured to read the processed hyperspectral image from the memory component and display the processed hyperspectral image on the display.

In some embodiments, the present invention features a hyperspectral medical imaging system for imaging a tissue. The system may comprise a hyperspectral sensor. The system may further comprise a transmissive optical component. The transmissive optical component may comprise a polarizer. The system may further comprise a lens or series of lenses, prisms, diffraction gratings, filters; both tunable and non-tunable, slits, mirrors, and interferometric components. The system may further comprise a light source emitting broadband light from about 400 nm to about 2500 nm. The system may further comprise a movable support boom. The hyperspectral sensor may be operatively coupled to the moveable support boom. The system may further comprise a display. The system may further comprise a computing device. The computing device may be operatively connected to the display and the hyperspectral sensor. The computing device may comprise a processor capable of executing computer-readable instructions and a memory component comprising computer-readable instructions.

The light source may be configured to emit incident broadband light. The incident broadband light may reflect off the tissue as reflected light. Alternatively, the light source may be positioned behind the tissue specimen to allow for imaging of the transmission spectrum from the sample. The transmissive optical component may be configured to transmit the reflected and/or transmitted light such that the reflected and/or transmitted light strikes the hyperspectral sensor. The hyperspectral sensor may be configured to detect a property of the reflected and/or transmitted light. The hyperspectral sensor may be further configured to capture a raw hyperspectral image. The hyperspectral sensor may be further configured to transmit the raw hyperspectral image to the computing device. The computing device may be configured to process the raw hyperspectral image and produce a processed hyperspectral image. The computing device may be further configured to transfer the processed hyperspectral image to the memory component. The computing device may be further configured to read the processed hyperspectral image from the memory component and display the processed hyperspectral image on the display.

In some embodiments, the present invention features a method of producing a hyperspectral medical image. The method may comprise illuminating an in vivo or ex vivo tissue specimen with a broadband light source emitting light from about 400 nm to about 2500 nm. The method may further comprise obtaining a scout image of the tissue specimen using a hyperspectral sensor to increase a hyperspectral image quality by ensuring the tissue specimen is centered within a field of view of the hyperspectral sensor and by adjusting at least one of a gain level, light intensity, a contrast sensitivity, exposure length, aperture size, or polarizer orientation of the hyperspectral system such that one or more spectral waveband is well-scaled across a hyperspectral image; obtaining the hyperspectral image by capturing a reflectance spectrum of a face of the tissue specimen, a transmission spectrum of light passing through the tissue specimen, or a combination thereof by the hyperspectral sensor. The method may further comprise saving the hyperspectral image as a datafile.

The method may further comprise transferring the datafile comprising the hyperspectral image to a computing device. The computing device may comprise a processor capable of executing computer-readable instructions and a memory component comprising computer-readable instructions for processing the hyperspectral image. The method may further comprise obtaining an endmember from at least one of the hyperspectral images or an endmember library. The endmember may be a reference candidate spectrum. The endmember may represent in the wavelength bands a material in the field of view used to collect the hyperspectral image.

The method may further comprise using the computing device to process the datafile to unmix the hyperspectral image by expressing a recorded spectrum as a linear or nonlinear combination of a set of endmembers. The method may further comprise using the computing device to generate a color-coded image from an unmixed hyperspectral image. The method may further comprise displaying the color-coded image on a display. The color-coded image may contain a colored pixel. The color of that pixel is determined by the coefficients in the linear or non-linear combination of endmembers representing the recorded spectrum. Each endmember may correspond to a tissue type present in a tissue specimen location in or on the tissue specimen.

In some embodiments, the present invention features an image acquisition system that controls or operates the hyperspectral sensor. In some embodiments, depending on the level of detail needed for a given application, the image acquisition system allows the user to adjust the focus, contrast, exposure time, gain, and other image acquisition parameters and displays a scout image, i.e., a preview image before capturing the final hyperspectral image to be used for clinical analysis. In some embodiments, this allows the user to determine how well subjects in the hyperspectral sensor's field of view are in focus and/or how well the various spectral bands are being captured.

In some embodiments, the unmixing process may further comprise obtaining at least one endmember from at least one of the hyperspectral images or an endmember library. The at least one endmember may be a reference candidate spectrum. The endmember may represent in the wavelength bands a material in the field of view used to collect the hyperspectral image. Unmixing may estimate, approximate, and/or represent a recorded pixel spectrum as a linear combination of endmembers. The pixel spectrum may represent a reflectance spectrum and/or transmission spectrum in a field of view of the at least one sensor pixel. Unmixing may further comprise comparing the pixel spectrum to the set of endmembers. Unmixing may further comprise estimating the composition of a material in the field of view of the at least one sensor pixel by comparing the pixel spectrum corresponding to the at least one sensor pixel to the set of endmembers and constructing a combination of at least one endmember that approximates the pixel spectrum recorded by the at least one sensor pixel represented as the image pixel in the hyperspectral image. Unmixing may further comprise expressing each image pixel in the hyperspectral image as a combination of at least one endmember.

Each endmember may be associated with an endmember coefficient that represents a proportion of the tissue specimen that corresponds to the at least one endmember present in the tissue specimen location in or on the tissue specimen. Each of the endmember coefficients may thus correspond to the proportion of tissue type present in the tissue specimen location in or on the tissue specimen. Each of the endmembers may be assigned to an image pixel color. Each image pixel in the hyperspectral image may assume a combined image pixel color on the display corresponding to the relative proportion of each endmember and tissue type present in the tissue specimen location in or on the tissue specimen. Each pixel may be represented as a linear combination of endmember spectra.

In some embodiments, the present invention may be used to construct an (r, g, b) color as three (linear) combinations of endmember abundances, in which the coefficients determine the (r, g, b) components. In some embodiments, a method of the present invention may further comprise coloring each pixel based on endmember abundances. There are two constructions involved in the algorithm: unmixing and heatmapping. Unmixing constructs a combination of endmembers to approximate a recorded pixel spectrum; the coefficients in that combination are abundances. Heatmapping constructs a display value for each pixel by using the pixels' endmember abundances to produce 3 display colors, r, g, and b. For example, if the relative abundances of healthy, fatty, and cancerous tissues in a pixel are (0.7, 0.1, 0.2), then r=0.2, g=0.7, and b=0.2. In some embodiments, if there are ten materials present in an image, each should be mapped to a distinct color. Generally, heatmapping assigns an (r, g, b) triplet to each set of abundance coefficients. Each pixel is assigned a color triplet according to the abundance of materials in that pixel.

In some embodiments, the method of the present invention may further comprise selecting at least one endmember according to the following steps. The steps may comprise randomly or pseudo-randomly selecting a number (k) of at least one pixel (p) to represent a set of at least one endmembers, each of the at least one pixel representing a pixel cluster. The pixel cluster may comprise pixels nearest to one of the at least one endmembers as compared to other endmembers. The steps may further comprise assigning each of the at least one pixel with a cluster identifier corresponding to the pixel cluster the at least one pixel is a part of. The steps may further comprise labeling each pixel of the pixel cluster with its nearest endmember. The steps may further comprise repeating the aforementioned steps for all pixels of the pixel clusters. The steps may further comprise computing a mean spectrum for a pixel cluster. The steps may further comprise calculating a mean cluster change for the pixel cluster. The mean cluster change may be the magnitude of the change between the current mean spectrum of the pixel cluster and the previous mean spectrum of the same pixel cluster. The steps may further comprise finding a maximum magnitude of the mean cluster change among the calculated mean cluster changes. The steps may further comprise repeating the aforementioned steps until the maximum mean cluster change is at or below a threshold value. The steps may further comprise utilizing each resulting mean spectrum as an endmember for unmixing. The endmember may be a reference candidate spectrum and the endmember represents in the wavelength bands a material in the field of view used to collect the hyperspectral image.

In some embodiments, the method of the present invention may further comprise extracting a spectrum from areas of a tissue sample with known properties (e.g. healthy, cancerous, fatty, etc.) based on pathology results. The method may further comprise obtaining a pixel spectrum using a hyperspectral sensor for a point on the reference tissue. The method may further comprise appending a tag to the pixel spectrum of the reference tissue to create a tagged pixel spectrum. The tag may indicate the reference tissue's tissue type. The method may further comprise storing the tagged pixel spectrum corresponding to the reference tissue in an endmember library. The method may further comprise obtaining a pixel spectrum using a hyperspectral sensor for a point on an in vivo or ex vivo clinical tissue. The method may further comprise determining an unknown tissue type present in the clinical tissue by estimating abundances of reference spectra.

In some embodiments, the scout image may be used to increase a hyperspectral image quality by adjusting at least one of the following parameters of the hyperspectral imager: aperture, exposure time, ISO, light source intensity, or rotation of a polarizer or other transmissive or semi-transmissive optical element. In some embodiments, the method of the present invention may further comprise obtaining increasingly low spectral resolution data from the clinical tissue and reconstructing an approximated spectral profile by interpolating spectral data between data points of the low spectral resolution data obtained in the aforementioned steps using spectral data present in the endmember library.

In some embodiments, the method of the present invention may further comprise selecting a distinctive spectral feature indicative of the presence of a particular tissue type. The method may further comprise limiting the analysis of a hyperspectral image to those features within an analyzed spectral range of the distinctive spectral feature selected. Limiting analysis according to the aforementioned step may improve the processing time of the hyperspectral image.

In some embodiments, the distinctive spectral feature indicative of the presence of a particular tissue type may comprise a spectral inflection point. In some embodiments, the distinctive spectral feature indicative of the presence of a particular tissue type may comprise a unique combination of spectral inflection points. As a non-limiting example, a given tissue may have three spectral peaks. One of the peaks may have a lower intensity relative to the other two peaks, which may be a defining characteristic of the given tissue, as compared to a second tissue which may have three spectral peaks in which all three peaks have relatively equal intensities, or a third tissue which may have three spectral peaks in which one spectral peak is shifted to a longer or shorter wavelength as compared to the analogous peak present in the first two tissues. In some embodiments, the analyzed spectral range is narrower than the complete spectral range of the pixel spectrum obtained by the hyperspectral sensor. In some embodiments, the spectral inflection point is determined by finding a derivative of the pixel spectrum.

In some embodiments, the present invention features a method of performing surgery with the systems of or methods of the present invention. In some embodiments, said method of performing surgery may comprise obtaining a raw hyperspectral image of an excised tissue. The method may further comprise producing a processed hyperspectral image of the excised tissue. The method may further comprise displaying the processed hyperspectral image on a display to identify regions of spectral interest that may correlate with pathology. As a non limiting example, by detecting residual tumor on the surface of an excised sample, an operator may interpret the processed hyperspectral image on the display to determine if the excision of a tumor is complete or incomplete. The operator may thus make surgical adjustments accordingly.

In some embodiments, the present invention features a method of performing surgery with the systems or methods of the present invention. The method may comprise obtaining a raw hyperspectral image of an in vivo tissue. The method may further comprise producing a processed hyperspectral image of the in vivo tissue. The method may further comprise displaying the processed hyperspectral image on a display. An operator may interpret the processed hyperspectral image on the display to determine if the excision of a tumor in or around the in vivo tissue is complete or incomplete. The operator may thus make surgical adjustments accordingly.

In some embodiments, the present invention features a method of performing surgery with the systems or methods of the present invention. The method may comprise obtaining a raw hyperspectral image of an in vivo tissue. The method may further comprise producing a processed hyperspectral image of the in vivo tissue. The method may further comprise displaying the processed hyperspectral image on a display. An operator may interpret the processed hyperspectral image on the display to determine the location of a tumor in or around the in vivo tissue. The operator may thus make surgical decisions accordingly. In some embodiments, the processed hyperspectral image displayed on the display may be manipulable by an operator.

In some embodiments, the present invention features a method of performing core needle biopsy with the systems or methods of the present invention. The method may comprise obtaining a raw hyperspectral image of an excised core needle biopsy tissue specimen. The method may further comprise producing a processed hyperspectral image of excised core needle biopsy tissue. The method may further comprise displaying the processed hyperspectral image on a display. An operator may interpret the processed hyperspectral image on the display to determine if there are multiple unique spectral profiles present, or if characteristic spectral profiles are present. The operator may thus make surgical decisions accordingly, for example to obtain another core or complete the procedure. In some embodiments, the processed hyperspectral image displayed on the display may be manipulable by an operator.

In some embodiments, the present invention features a method of performing specimen grossing, sectioning, processing, or analysis. The method may comprise obtaining a raw hyperspectral image of an ex vivo tissue or tissues. The method may further comprise producing a processed hyperspectral image of the ex vivo tissue or tissues. The method may further comprise displaying the processed hyperspectral image on a display. An operator may interpret the processed hyperspectral image on the display to determine the at least one unique spectral profile on the ex vivo tissue or tissues. For the purposes of tissue grossing, whereby an operator is tasked with sampling regions of tissue from a larger block for subsequent analysis, the operator may make decisions about which areas to source samples from based on the processed hyperspectral image. In some embodiments, the processed hyperspectral image displayed on the display may be manipulable by an operator.

In some embodiments, the incident broadband light may comprise a wavelength of about 400 nm to about 2500 nm. In some embodiments, the incident broadband light may comprise a wavelength of about 800 to 1700 nm. In some embodiments, the incident broadband light may comprise a wavelength of about 400 to 900 nm. In some embodiments, the incident broadband light may comprise a wavelength of about 200 to 700 nm. In some embodiments, the incident broadband light may comprise a wavelength of about 1000 to 2500 nm.

As non limiting examples, in some embodiments, the hyperspectral system may operate at a spectral resolution of around 5-8 nm with a full width at half maximum (FWHM) of around 1-2 nm. In some embodiments, the hyperspectral system may operate at a spectral resolution of 5-20 nm with a variable FWHM. In some embodiments the hyperspectral system may operate at a spectral resolution of around 5-10 nm with a FHWM of 8-10 nm. In some embodiments the number of spectral bands may be 50-60 bands. In some embodiments, the number of spectral bands may be 200 to 600 bands. In some embodiments, the hyperspectral sensor may operate at a spectral resolution of about 100 bands. In some embodiments, the hyperspectral sensor may operate at a spectral resolution of about 200 bands. In some embodiments, the hyperspectral sensor may operate at a spectral resolution of about 500 bands. In some embodiments, the hyperspectral sensor may operate at a spectral resolution of more than 500 bands.

In some embodiments, the tissue may contain a breast tumor. In some embodiments, the tissue may be affected by diabetic retinopathy. In some embodiments, the tissue may be affected by a retinal bleed. In some embodiments, the tissue may be affected by retinal neovascularization. In some embodiments, the tissue may be affected by choroidal neovascularization. In some embodiments, the tissue may be affected by a hereditary retinal condition. In some embodiments, the tissue may be affected by hereditary retinal degeneration. In some embodiments, the tissue may be affected by an inherited retinal disease. In some embodiments, the tissue may be affected by retinal edema.

In some embodiments, the tissue may be affected by central serous retinopathy. In some embodiments, the tissue may be affected by central serous chorioretinopathy. In some embodiments, the tissue may be affected by Irvine Gass. In some embodiments, the tissue may be affected by retinal dystrophy. In some embodiments, the tissue may be affected by retinal vasculitis. In some embodiments, the tissue may be affected by retinal necrosis. In some embodiments, the tissue may be affected by age-related macular degeneration (AMD). In some embodiments, the tissue may be injected with a gene therapy product and the system is used to visualize the spread of the gene therapy product through the tissue. In some embodiments, the tissue may be an ocular tissue and the ocular tissue may be injected via at least one of an intravitreal injection or a subretinal injection. In some embodiments, the present invention may be used to detect signatures of at least one of perfusion or cell death.

In some embodiments, the tissue may be a tissue undergoing surgical intervention. In some embodiments, the tissue may be a bowel tissue undergoing surgical resection. In some embodiments, the present invention may be used to detect a border of necrotic bowel. In some embodiments, the present invention may be used to detect the vitality of an anastomosis. In some embodiments, the present invention may be used to identify lymphatic tissue. In some embodiments, the lymphatic tissue may be a lymph node. In some embodiments, the tissue may be a thyroid tissue. In some embodiments, the present invention may be used to differentiate thyroid tissue from parathyroid tissue. In some embodiments, the present invention may be used to differentiate a healthy thyroid tissue from a diseased thyroid tissue.

In some embodiments, the tissue may be a parathyroid tissue. In some embodiments, the present invention may be used to differentiate thyroid tissue from parathyroid tissue. In some embodiments, the present invention may be used to differentiate a healthy parathyroid tissue from a diseased parathyroid tissue. In some embodiments, the present invention may be used to identify a neurovascular bundle. In some embodiments, the neurovascular bundle may comprise a facial nerve. In some embodiments, the neurovascular bundle may comprise a recurrent laryngeal nerve.

In some embodiments, the tissue may be an esophageal tissue. In some embodiments, the present invention may be used during an esophagectomy. In some embodiments, the present invention may be used to determine the margin of an esophageal tumor. In some embodiments, the present invention may be used to identify an intraluminal change present in the esophageal tissue. In some embodiments, the intraluminal change may be an intraluminal change associated with at least one of the following: an esophageal dysplasia, an esophageal neoplasm, a cancerous lesion, or a precancerous lesion.

In some embodiments, the tissue may be a colorectal tissue. In some embodiments, the present invention may be used during a colorectal surgery. In some embodiments, the present invention may be used to determine the margin of a colorectal tumor. In some embodiments, the present invention may be used to identify an intraluminal change present in the colorectal tissue. In some embodiments, the intraluminal change may be an intraluminal change associated with at least one of the following: a colorectal dysplasia, a colorectal neoplasm, a cancerous lesion, or a precancerous lesion.

In some embodiments, the present invention may be used to determine a transition between at least two burns of different severities. In some embodiments, the present invention may be used to identify the transition between a first-degree burn and a second-degree burn. In some embodiments, the present invention may be used to identify the transition between a second-degree burn and a third-degree burn. In some embodiments, the present invention may be used to identify the transition between a first-degree burn and a third-degree burn.

In some embodiments, the present invention may be used to identify a transition between necrotic tissue and healthy tissue. In some embodiments, the present invention may be used to identify a transition between infected tissue and healthy tissue. In some embodiments, the present invention may be used to identify at least one of the following characteristics of an ulcer: a stage, a grade, a perfusion status, or an area of necrosis. In some embodiments, the present invention may be used to identify at least one of the following characteristics of a wound: a stage, a grade, a perfusion status, or an area of necrosis. In some embodiments, the present invention may be used to identify at least one of the following characteristics of a chronic wound: a stage, a grade, a perfusion status, or an area of necrosis.

In some embodiments, the present invention may be used to determine the margin of a solid tumor. In some embodiments, the present invention may be used to determine the margin of a bladder tumor. In some embodiments, the present invention may be used to determine the margin of a breast tumor. In some embodiments, the present invention may be used to determine the margin of a liver tumor. In some embodiments the present invention may be used to determine the margins of a biliary tumor. In some embodiments, the present invention may be used to determine the margin of a colorectal tumor. In some embodiments, the present invention may be used to determine the margin of the colorectal tumor on an excised ex vivo tissue. In some embodiments, the present invention may be used to determine the margin of the colorectal tumor on an in vivo tissue. In some embodiments, the present invention may be used to determine the margin of a uterine tumor. In some embodiments, the present invention may be used to determine the margin of an endometrial tumor. In some embodiments, the present invention may be used to determine the margin of a cervical tumor. In some embodiments, the present invention may be used to determine the margin of a prostate tumor. In some embodiments, the present invention may be used to determine the margin of a renal tumor. In some embodiments, the present invention may be used to determine the margin of a liver tumor. In some embodiments, the present invention may be used to determine the margin of a bile duct tumor. In some embodiments, the present invention may be used to determine the margin of a lung tumor. In some embodiments, the present invention may be used to determine the margin of the lung tumor on an excised ex vivo tissue. In some embodiments, the present invention may be used to determine the margin of the lung tumor on an in vivo tissue. In some embodiments, the present invention may be used to determine the margin of a skin tumor.

In some embodiments, the skin tumor may comprise at least one of the following: a basal cell carcinoma, a squamous cell carcinoma, or a melanoma. In some embodiments, the present invention may be used to determine the margin of the skin tumor on an excised ex vivo tissue. In some embodiments, the present invention may be used to determine the margin of the skin tumor on an in vivo tissue.

In some embodiments, the present invention may be used to determine the margin of a pancreatic tumor. In some embodiments, the present invention may be used to determine the margin of the pancreatic tumor on an excised ex vivo tissue. In some embodiments, the present invention may be used to determine the margin of the pancreatic tumor on an in vivo tissue. In some embodiments, the present invention may be used to determine the margin of a thyroid tumor. In some embodiments, the present invention may be used to determine the margin of a parathyroid tumor.

In some embodiments, the present invention may be used to determine the margin of an intracranial tumor. In some embodiments, the intracranial tumor may comprise at least one of the following: a metastatic tumor, a meningioma, a glioblastoma, or an astrocytoma. In some embodiments, the present invention may be used to determine the margin of the intracranial tumor on an excised ex vivo tissue. In some embodiments, the present invention may be used to determine the margin of the intracranial tumor on an in vivo tissue. In some embodiments, the present invention may be used to determine the margin of an oropharyngeal tumor. In some embodiments, the present invention may be used to differentiate a healthy adrenal tissue from a diseased adrenal tissue.

In some embodiments, the present invention may be used in conjunction with core needle biopsy to identify if the target in question has been sampled. In some embodiments, the core needle biopsy may be drawn from a breast and the present invention may be used to determine if some combination of healthy and diseased breast tissue is present in the sample. In some embodiments, the core needle biopsy may be drawn from the liver, and the present invention may be used to determine if some combination of healthy and diseased liver tissue is present in the sample. In some embodiments the core needle biopsy may be drawn from the retroperitoneum and the present invention may be used to determine if some combination of healthy and diseased retroperitoneal tissue is contained within the sample. In some embodiments the core needle biopsy may be drawn from the lung and the present invention may be used to determine if some combination of healthy and diseased lung tissue is present in the sample. In some embodiments the core needle biopsy may be drawn from a lymph node and the present invention may be used to determine if some combination of healthy and diseased nodal tissue is present within the sample.

In some embodiments, the present invention may be used to determine the location of spectrally distinct regions on the surface of ex vivo tissue blocks being prepared for sectioning or grossing. In some embodiments, the present invention may be used to aid pathologists in identifying tissue types and histopathologic features on slides of prepared tissue.

In some embodiments, the display may comprise a projector. The projector may project the color-coded image onto the in vivo or ex vivo tissue specimen. In some embodiments, the display may display a 3D reconstruction of an excised tissue. An operator of the system or a user of the method may specify a face of the excised tissue that is being imaged before or during a time in which the hyperspectral image is captured. A finished 3D pseudocolored image may be generated showing the face of the excised tissue. Said 3D pseudocolored image may be manipulated by the operator of the system or the user of the method. The 3D pseudocolored image of the excised tissue may be virtually mapped onto or into a resection pocket hyperspectral image displayed on the display using feature co-registration. A first tumor feature on the 3D pseudocolored image of the excised tissue corresponding to a first tumor feature location on the excised tissue may line up with a second tumor feature on the resection pocket hyperspectral image corresponding to a second tumor feature location on the resection pocket.

In some embodiments, the hyperspectral medical image may be processed in real-time such that the color-coded image displayed on the display is displayed and updated in five seconds or less. In some embodiments, the present invention may produce a hyperspectral medical image in real-time such that the color-coded image displayed on the display is displayed and updated in five seconds or less. In some embodiments, the present invention may utilize real-time computing. In some embodiments, the present invention may be used to identify at least one of any spectral correlates of disease or spectrally distinct regions on excised biopsy or pathology specimens.

The light source may comprise light-emitting diodes (LEDs), lasers, halogen lamps, mercury lamps, or a combination thereof. The transmissive optical component may comprise lenses, filters, slits, diffraction gratings, interferometers, polarizers, beamsplitters, prisms, optical flats, windows, mirrors, retroreflectors, wave plates, or a combination thereof.

### EXAMPLE

The following are non-limiting examples of the presently claimed invention:

A system was set up for reflectance imaging. Tissue was placed on the stage, the lights turned on, and the sample was imaged. For a cabinet-style system, the system comprised an outer shell with attached wheels, a display, an integrated computer, a keyboard, and a mouse. Inside the shell, the system comprised a hyperspectral camera mounted at the top with front optics including a polarizer and 50 mm C-Mount lens, spectrograph, and InGaAs sensor. Broadband halogen lights were built into the top of the system and shined down onto the stage. The inner lining of the box was a matte white coating to diffuse light evenly. The stage itself translated linearly to allow for line-scan image acquisition. Consequently, the enclosure was only 3-sided and open on one side to allow the stage to slide out at maximum excursion. The computer and DAQ board along with operating software controlled the image acquisition parameters (light intensity, ISO, aperture, integration time). Scan speed was also adjustable (slower translation of stage->more lines in the scan->higher spatial resolution). Lenses were capable of being switched out and the polarizer was capable of being adjusted as needed for a given image.

The process of image acquisition started with placing samples on the stage, obtaining a quick scout image, and repeating that as needed by adjusting image parameters. The display showed basic light curves during this portion to show if all the wavelength bands were well exposed or if some were washed out. The polarizer could be adjusted, the lights could be turned down, or the exposure time could be decreased to minimize the effect of glare or very bright pixels as the tissue surface was often wet. Once satisfied, the parameters were fixed and a full scan took place. This took around 40 seconds. Then, a processing stage took place as described in the above sections. Finally, the output was black and white images that highlighted single spectral profiles/single tissue types, as well as pseudocolor maps that synthesized those into a single overlapped false color image.

A system was set up for transmissive imaging. The objective in this version was to sample the light passing through tissue rather than reflecting off the surface. This was only useful for imaging thin specimens, most typically at higher magnification, such as core needle samples (1-3 mm wide/thick and several inches long), and microscopic analysis (10-100× zoom, looking at slides of prepared tissue 5-10 um thick at high magnification).

Transmission spectroscopy was on a benchtop system. The system may be capable of both reflectance imaging and transmission imaging. In that case, a light source is placed above the sample in the reflectance mode, and in the transmission mode, the light source is placed behind the sample.

For the transmission system, the stage was an optically transparent medium. The samples for transmission imaging needed to be compressed. The front and back surfaces of the tissue were made to be as flat as possible. Surface irregularities cause light to scatter unevenly rather than just pass through the sample, so an adjustable tissue holder was implemented to address this, comprising two flat pieces of optical glass that sandwiched the specimen. The system further comprised an adjustment mechanism to allow the gap between the flat pieces to be 1 mm, 2 mm, 3 mm, etc. The gap was sized to the sample width to make sure the surfaces were flat.

Once the sample was in the imaging case, it was positioned in front of the light (i.e. between the light and the camera) so that it was backlit. The light source was a broadband light source. The light passed through the specimen into the camera as described in the above sections and was imaged and processed according to the above sections to generate similar color coded outputs highlighting spectral features of the tissue.

A system for resection pocket imaging was developed. This iteration of the system effectively did the same reflectance imaging as described above but with the camera attached to a support arm that was capable of being positioned with respect to the resection pocket. This allowed surgeons to make decisions prior to resecting tissue. The system comprised optics, a spectrograph, and a sensor along with the lights that all form one unit at the end of the boom. The lights were adjustable with respect to the pocket, like spotlights. The optics were configured with a fixed focal length and wide field view or allowed users to select such properties using an adjustable combination of lenses controlled by a computer.

The present invention comprised a spectral library comprising a set of endmembers. The spectral library was typically saved as a database on a computer., using XML as a file format. Other database structures such as SQL and postgreSQL may be used. The spectral library included a method for adding, modifying, interrogating, and deleting entries, adding annotations, and displaying data saved in the library. Endmembers in a spectral library were typically read from the database as part of an image unmixing process. Spectra were identified as endmembers and added to a spectral library via several methods described below.

Any endmember was capable of being added to a spectral library. Addition to a library was determined by a user or automatically. A set of endmembers was used to unmix a hyperspectral image. An example of endmember identification was direct sampling, comprising identifying an endmember by sampling a hyperspectral image. Sampling comprised identifying a location in an image and computing the average spectrum over a neighborhood. The neighborhood size was arbitrarily large but nonzero.

Endmembers were also able to be identified by a clustering algorithm, in which each cluster was represented by a single spectrum and may be added to a spectral library or used for unmixing. This process began by selecting k pixels, called means, from the image and defining a distance measure on the image, the squared Euclidean distance. The process further comprised choosing a number, x, which determined the endpoint of the clustering algorithm. For each pixel, the distance to each mean was computed and each pixel was assigned to the nearest mean. New means were then computed. For each mean, the mean was recomputed as the average of all pixels assigned to that mean, and the magnitude of the change in each mean was computed, with c; defined as the magnitude of the i^{th} mean change. The previous two steps were repeated until the maximum of the set {c} fell below x. When the algorithm stopped, any subset of the set of means was able to be used as endmembers.

A coordinate system in the hyperspectral data space was identified so that the data was transformed relative to the set of basis vectors which captured the largest variation in the data. M was defined as a matrix of hyperspectral data. The transpose, M^{T} of M was computed, along with the covariance matrix K=M^{T}M. Then, the eigenvalues and eigenvectors of K were computed. A subset of the eigenvectors was able to be chosen and used as endmembers. Typically, the eigenvectors with the k greatest eigenvalues were chosen.

Unmixing: Two alternate methods of unmixing were implemented, each of which took a set of endmembers and produced a set of abundance arrays. The set of abundance arrays were used to construct abundance maps and heat maps.

In the first method, a set of endmembers were identified. This occurred with direct sampling, retrieval from a spectral library, data clustering, principal component analysis, as above, or any other method. Each pixel was a vector, p, of recorded reflectance amplitudes. An estimate, q, of p was constructed as a linear combination of endmembers. S was defined as a matrix of endmembers spectra. Then, q=Sa. The coefficient vector, a, was called an abundance vector, subject to the requirement Σaⱼ=1. For each pixel, a was computed as the vector which minimized ||p−q||², where || . . . ||denotes Euclidean distance. This was given by a=(S^{T}S)⁻¹S^{T}p. Therefore, for each pixel, a vector of endmember abundances was generated. This set of abundance vectors was used to create abundance maps which were consolidated into a heatmap.

In the second method, for each pixel, the error in the linear approximation was computed: e=p−q=p−Sa, subject to the requirement Σaⱼ=1. A weight matrix W whose diagonals are the reciprocals, 1/σ², of the error variances, σ², and whose off diagonals are zero was defined: W={Wᵢⱼ |Wᵢⱼ=1/σ² for i=j and Wᵢⱼ=0 otherwise}. The abundance vector was computed as a=(S^{T}WS)⁻¹S^{T}Wp. As above, for each pixel, there was a vector of endmember abundances. This set of abundance vectors was used to create abundance maps that were consolidated into a heatmap.

Abundance Maps and Heat Maps: For each pixel, a vector of endmember abundances, a, whose length is the number, k, of endmembers used in unmixing was determined. An integer j≤k was chosen. For each pixel, the abundance aⱼ was selected. The abundance map was an array of values {aᵢ}, one for each pixel in the image. The abundance map was represented as a two-dimensional grayscale image of abundance values. k images were constructed, each of which represented the relative abundance of an endmember at each pixel location in an image. The abundance values were globally rescaled by a uniform factor for image display. The greyscale abundance images were saved to a computer file and the images were displayed with the display system.

Heatmap: Three functions, f_{R}, f_{G}, and f_{B}: R^{k}→R were constructed that mapped each abundance vector to a real number. For each pixel, a color c was constructed in R³: c=(f_{R}(a), f_{G}(a), f_{B}(a)). A heatmap was an array {cᵢ}. The heatmap was represented as an RGB color image. The display color at a pixel was given by the three-dimensional color array at that pixel location. The image was saved to a computer file and displayed through the display system.

The computing device (otherwise referred to as a computing system) may include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from a 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on a computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer-readable medium may be transmitted using any appropriate medium, including but not limited to wireless, wireline, optical fiber cable, RF, Bluetooth, storage media, computer buses, etc., or any suitable combination of the foregoing. Computer program code for carrying out operations for aspects of the present disclosure may be written in any combination of one or more programming languages, including an object-oriented programming language such as Java, Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft® .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

As used herein, the term “about” refers to plus or minus 10% of the referenced number. As used herein, the term “population” refers to one or more. As used herein, the terms “a,” “an,” “the,” and “said” include both singular and plural uses, and thus include one or more items. For example, the phrases “a microprocessor,” “the microprocessor,” and “said microprocessor” all encompass one or more microprocessors.

In some embodiments, the figures presented in this patent application are drawn to scale, including the angles, ratios of dimensions, etc. In some embodiments, the figures are representative only and the invention is not limited by the dimensions of the figures. In some embodiments, descriptions of the inventions described herein using the phrase “comprising” includes embodiments that could be described as “consisting essentially of” or “consisting of”, and as such the written description requirement for providing one or more embodiments of the present invention using the phrase “consisting essentially of” or “consisting of” is met.

What is claimed:

1. A hyperspectral medical imaging system for imaging a tissue, the hyperspectral medical imaging system comprising:
a. a light source configured to emit incident light, the incident light reflecting off the tissue as reflected light, passing through the tissue as transmitted light, or a combination thereof;
b. a transmissive optical component configured to transmit the reflected light, the transmitted light, or the combination thereof;
c. a hyperspectral sensor configured to detect a property of the reflected light, the transmitted light, or the combination thereof transmitted through the transmissive optical component, the hyperspectral sensor further configured to capture a raw hyperspectral image and transmit the raw hyperspectral image to a computing device;
d. the computing device operatively connected to the hyperspectral sensor, the computing device comprising a processor capable of executing computer-readable instructions and a memory component comprising the computer-readable instructions, wherein the computing device is configured to process the raw hyperspectral image to produce a processed hyperspectral image, and transfer the processed hyperspectral image to the memory component; and
e. a display operatively connected to the computing device, wherein the computing device is further configured to read the processed hyperspectral image from the memory component and display the processed hyperspectral image on the display;
wherein the raw hyperspectral image is processed in real-time such that the processed hyperspectral image displayed on the display is displayed and updated in five seconds or less.

2. The system of claim 1, wherein the processed hyperspectral image displayed on the display is manipulable by an operator.

3. The system of claim 1, wherein the incident light is from about 400 nm to about 2500 nm.

4. The system of claim 1, wherein the transmissive optical component comprises a polarizer, an objective lens, a field correcting lens, or a combination thereof.

5. The system of claim 1, wherein the hyperspectral sensor is configured to produce high resolution spectral data in a range of 500 to 2500 nm.

6. The system of claim 1 further comprising a spectrograph disposed optically in-line with the transmissive optical component and the hyperspectral sensor, the spectrograph comprising an opening configured to accept the reflected light, the transmitted light, or the combination thereof, and a dispersive element configured to spread a spectra of the reflected light, the transmitted light, or the combination thereof into a plurality of spectral bands focused towards the hyperspectral sensor.

7. A method of producing a hyperspectral image comprising:
a. illuminating an in vivo or ex vivo tissue specimen with a light source;
b. obtaining a scout image of the tissue specimen using a hyperspectral sensor to increase a hyperspectral image quality;
c. obtaining the hyperspectral image by capturing a reflectance spectrum of a face of the tissue specimen, a transmission spectrum of light through the tissue, or a combination thereof using the hyperspectral sensor;
d. saving the hyperspectral image as a datafile to a computing device, the computing device comprising a processor capable of executing computer-readable instructions and a memory component comprising computer-readable instructions for processing the hyperspectral image obtained by the hyperspectral sensor;
e. obtaining at least one endmember from at least one of the hyperspectral image or an endmember library, wherein the at least one endmember is a reference candidate spectrum, wherein the at least one endmember represents a material in wavelength bands used to collect the hyperspectral image;
f. using the computing device to process the datafile to unmix the hyperspectral image against the at least one endmember;
g. using the computing device to generate a color-coded image from an unmixed hyperspectral image; and
h. displaying the color-coded image on a display operatively connected to the computing device;
wherein the color-coded image contains one or more pixels whose color is determined by the at least one endmember.

8. The method of claim 7, wherein unmixing the hyperspectral image comprises:
a. obtaining a pixel spectrum recorded by at least one sensor pixel in the hyperspectral sensor which corresponds to an image pixel of a plurality of image pixels in the hyperspectral image, wherein the pixel spectrum represents a reflectance spectrum, a transmission spectrum, or a combination thereof in a field of view of the at least one sensor pixel;
b. comparing the pixel spectrum to the at least one endmember;
c. estimating a composition of a material in the field of view of the at least one sensor pixel by comparing the pixel spectrum corresponding to the at least one sensor pixel to the at least one endmember and constructing a combination of at least one endmember that approximates the pixel spectrum recorded by the at least one sensor pixel represented as the image pixel in the hyperspectral image; and
d. expressing each image pixel in the hyperspectral image as a combination of the at least one endmember that approximates the pixel spectrum recorded by the at least one sensor pixel represented as the image pixel in the hyperspectral image;
wherein each endmember comprising the combination of the at least one endmember represented in each image pixel of the hyperspectral image is associated with an endmember coefficient;
wherein each endmember coefficient represents a proportion of the tissue specimen that corresponds to at least one endmember present in a location in or on the tissue specimen;
wherein each endmember coefficient thus corresponds to a proportion of tissue type present in the location in or on the tissue specimen; and
wherein each image pixel in the hyperspectral image assumes an image pixel color on the display corresponding to a relative proportion of each endmember and tissue type present in the tissue specimen location in or on the tissue specimen.

9. The method of claim 7, wherein the method further comprises generating at least one endmember according to the following steps:
a. randomly or pseudo-randomly selecting a number of at least one pixel to represent a set of at least one endmember, each of the at least one pixel representing a pixel cluster, the pixel cluster comprising pixels nearest to one of the at least one endmembers as compared to other endmembers;
b. assigning each of the at least one pixel with a cluster identifier corresponding to the pixel cluster the at least one pixel is a part of;
c. labeling each pixel of the pixel cluster with its nearest endmember;
d. repeating steps b and c for all pixels selected in step a;
e. computing a mean spectrum for the pixel cluster;
f. calculating a mean cluster change for the pixel cluster, wherein the mean cluster change is a change between a current mean spectrum of the pixel cluster and a previous mean spectrum of the pixel cluster;
g. finding a magnitude of a maximum mean cluster change among the calculated mean cluster changes;
h. repeating steps e, f, and g until the magnitude of the maximum mean cluster change is at or below a threshold value; and
i. utilizing each mean spectrum as an endmember for unmixing;
wherein the at least one endmember is a reference candidate spectrum and the at least one endmember represents in the wavelength bands a material in the field of view used to collect the hyperspectral image.

10. The method of claim 7, wherein the method further comprises:
a. obtaining a reference tissue comprising a known tissue type;
b. obtaining a reference pixel spectrum using the hyperspectral sensor for at least one point on the reference tissue;
c. storing the reference pixel spectrum corresponding to the reference tissue in the endmember library;
d. obtaining a clinical pixel spectrum using the hyperspectral sensor for a point on an in vivo or ex vivo clinical tissue; and
e. determining abundances of clinical tissue types by comparing the clinical pixel spectrum to the reference pixel spectrum.

11. The method of claim 7, wherein the method further comprises:
a. obtaining a tissue sample;
b. obtaining a region-of-interest (ROI) pixel spectrum using the hyperspectral sensor for at least one ROI on the tissue sample;
c. storing the ROI pixel spectrum corresponding to the tissue sample in the endmember library as at least one ROI endmember; and
d. using the computing device to unmix the hyperspectral image against the ROI endmember;
wherein the at least one ROI endmember defines a tissue type of the at least one ROI.

12. The method of claim 7, wherein the method of producing the hyperspectral image is performed in real-time such that the color-coded image displayed on the display is displayed and updated in five seconds or less.

13. A hyperspectral medical imaging system for imaging a tissue, the hyperspectral medical imaging system comprising:
a. a light source emitting broadband light from about 400 nm to about 2500 nm, the light source configured to emit incident broadband light, the incident broadband light reflecting off the tissue as reflected light, passing through the tissue as transmitted light, or a combination thereof;
b. a polarizer configured to filter and transmit the reflected light, the transmitted light, or the combination thereof;
c. one or more optical components configured to focus and transmit the reflected light, the transmitted light, or the combination thereof to a spectrograph;
d. the spectrograph configured to separate the incident light into separate bands and focus them on a hyperspectral sensor;
e. the hyperspectral sensor configured to detect a property of the reflected light, the transmitted light, or the combination thereof, the hyperspectral sensor further configured to capture a raw hyperspectral image and transmit the raw hyperspectral image to a computing device;
f. the computing device operatively connected to the hyperspectral sensor, wherein the computing device comprises a processor capable of executing computer-readable instructions and a memory component comprising computer-readable instructions, wherein the computing device is configured to process the raw hyperspectral image to produce a processed hyperspectral image, and transfer the processed hyperspectral image to the memory component; and
g. a display operatively connected to the computing device the display and the hyperspectral sensor, wherein the computing device is further configured to read the processed hyperspectral image from the memory component and display the processed hyperspectral image on the display.

14. The system of claim 13, wherein the one or more optical components comprise one or more magnifying lenses, one or more filters, or a combination thereof.
